(19) United States
(12) Patent Application Publication
(10) Pub. No.: US 2009/0093890 A1
(43) Pub. Date: Apr. 9, 2009

(54) Precise control of orthopedic actuators

(76) Inventor: Daniel Gelbart, Vancouver (CA)

Correspondence Address: DAN GELBART, 4706 DRUMMOND DR, VANCOUVER, BC, V6T-1B4, CA

(21) Appl. No.: 11/905,771

(22) Filed: Oct. 4, 2007

(51) Int. Cl. A61F 2/48; A61B 17/52; A61B 17/58; A61N 2/00; A61H 1/00; A61F 2/72; A61B 17/56

(52) U.S. Cl. 623/24; 600/9; 623/26; 606/90; 623/26; 601/2

(57) ABSTRACT

An actuator is implanted inside the body and attached to a bone. The actuator is controlled from outside the body using a changing magnetic field or creating mechanical motion of the tissue. The changing field is used to create power inside the actuator and precisely control its operation without requiring a transdermal connection. The power generated inside actuator can also be used to transmit data to the outside.

### FIELD OF THE INVENTION

The invention relates to the medical field and in particular to orthopedic surgery.

### BACKGROUND OF THE INVENTION

In certain orthopedic procedures, such as spine and bone straightening, bone lengthening, and increasing gap between bones it is required to exert a significant force over a long period of time (days to months). Because bones are brittle the procedure is done gradually, bringing the bones to the final position in many small increments. This concept is well known and is the basis of orthodontic teeth straightening. Unlike orthodontic work, where the adjustment is easily accessible, most orthopedic work of this kind requires either repeated surgeries or metal attachments protruding through the skin in order to apply, and periodically adjust, the forces externally. Penetrating the skin on an ongoing basis is undesirable because of infections, scars and many other reasons. Prior art attempting to adjust the spine without the above shortcomings relied on Shape Memory Alloys that can be heated from outside the body. Shape Memory alloys (SMA) are a Nitinol alloy (Nickel/Titanium) that change shape at a sharply defined temperature. Because the transition happens over a few degrees centigrade the operation is practically binary, with almost no ability to set the alloy accurately to a point between minimum and maximum deflection. This can be dangerous in orthopedics, as excessive bending can break the bone. Also, the operation is non-reversible. It is desired to have an actuator that can be placed inside the body, with no protruding parts or wires, and can be accurately adjusted over a wide range. In some cases feedback about the force or position is desired. In other cases bi-directional operation is desired. Another problem with SMA actuators is the fact that it is difficult to make them MRI compatible because a closed loop configuration is needed to heat them inductively. Such a single turn closed loop will heat up from the MRI RF field. It is desirable to have an MRI compatible actuator as MRI is used extensively in orthopedics.

In other orthopedic procedures it is desired to supply a support to reduce the load on the bones or cartilage. Such a support needs periodic adjustment as the bones may recede. It is also desirable to spread the load uniformly across the bone to prevent high stress points. One aspect of the invention is to provide such a support, adjustable from the outside of the body.

### SUMMARY OF THE INVENTION

An actuator is implanted inside the body and attached to a bone. The actuator is controlled from outside the body using a changing magnetic field or creating mechanical motion of the tissue. The changing field is used to create power inside the actuator and precisely control its operation without requiring a transdermal connection. The power generated inside actuator can also be used to transmit data to the outside.

### BRIEF DESCRIPTION OF THE DRAWINGS

FIG. 1 is a schematic diagram showing the actuator attached to a bone and controlled from the outside of the body.

FIG. 2 is a longitudinal section of an actuator based on a motor and a lead screw.

FIG. 3 is a cross section of an actuator based on heating and evaporating a liquid.

FIG. 4 is a cross section of an actuator based on a hydraulic press principle.

FIG. 5 is a schematic view of a ratcheting mechanism that can be used for moving an actuator in response to magnetic field pulses, ultrasound or by direct pressure on the tissue.

FIG. 6 is a section of a spine showing the use of a supporting actuator having a compliant interface to the bone.

### DETAILED DESCRIPTION

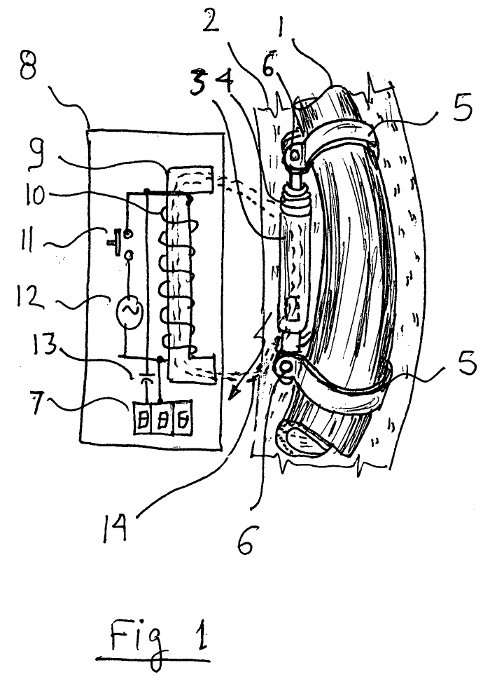

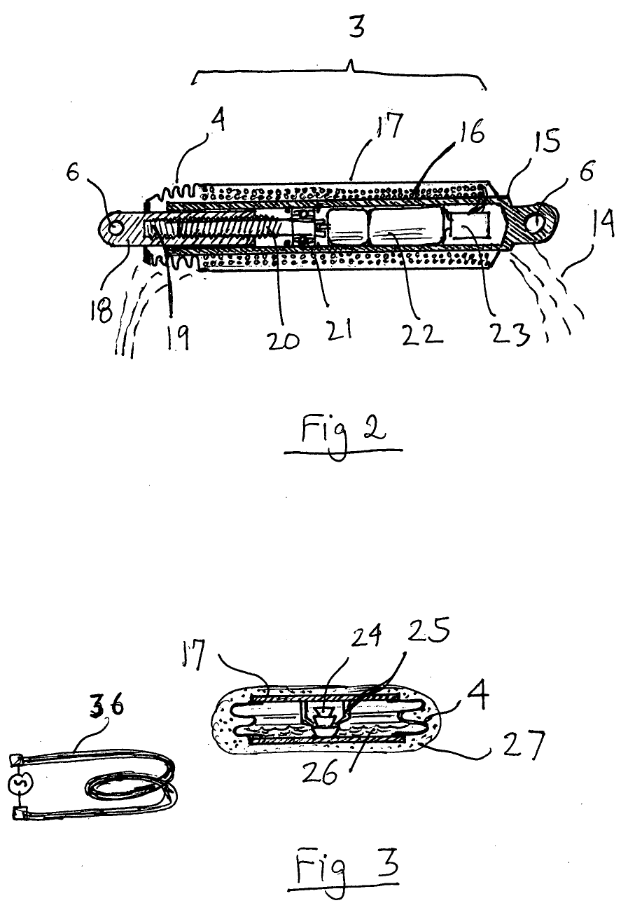

In this disclosure the word “actuator” means any device that can generate force or motion upon demand. The energy required for the motion is typically transmitted to the actuator from outside the patient's body, but actuators can have internal energy storage as well. For example, actuators can be powered by a battery, compressed spring, compressed gas, chemical reaction or any other form of energy storage. Also, the term “remotely operated” should be considered in the broadest sense. Any operation not requiring direct physical contact with the actuator is considered a “remote operation”, even if it involves pressing on the adjacent tissue or sending energy via a closely coupled transducer. FIG. 1 shows a typical application of an implanted actuator 3. It is temporarily attached to bone I by clamps 5 and pivots 6. Actuator 3 may be completely enclosed in tissue 2 and not require any transdermal (i.e. through the skin) connections. This greatly minimized risk of infections. Typically the actuator is hermetically sealed, with the moving part connected to the actuator body by metal bellows 4. Actuator 3 is activated by external control unit 8. In the preferred embodiment the control is achieved by inductively coupling coil 10 wound on ferromagnetic core 9 to a secondary coil located inside actuator. This is similar in operation to a regular transformer, with the exception of a larger air gap. In the simplest form, actuator 3 will move as long as it receives power from unit 8. Motion call be pulling, pushing, rotation etc. If desired, direction of motion can be switched by sending a command from unit 8 which is decoded by a logic circuit inside actuator. For example, the actuator can be made to respond only to long bursts of alternating current (AC) magnetic field, while a series of short bursts is decoded as a “reverse direction” command. While actuator is powered it can also send back, via inductive coupling or any other telemetry method, information on its position, force or any other desired information. A simple way to send information back is to superimpose a different AC frequency, carrying the desired information, on top of the inductive coupling. The power for generating the different AC frequency is derived from rectifying the AC signal received by the pick-up coil (secondary coil) inside the actuator, as will be explained later. Closing switch 1 1 connects AC power source 12 to coil 10 creating a magnetic field 14 coupling the secondary coil in actuator 3 to coil 10. If desired, the information transmitted back by actuator 3 at a different AC frequency is displayed on display 7. A filter blocks the AC power from source 12 from reaching display 7. The filter can be as simple as a capacitor 13 when the returned frequency is significantly higher than the transmitted frequency. While this example transmits the energy to actuator 3 by inductive coupling I is clear that other energy transmission mechanisms such as vibration (sub-sonic, sonic and ultrasonic) can be used either by converting them to electricity by a transducer inside actuator or by powering the actuator directly. Referring now to FIG. 2, actuator 3 has a coil 16 wound on a ferromagnetic body 15 and sealed by a hermetic metal enclosure 17. Plunger 18 is sealed by connecting it to enclosure 17 by a flexible metal bellows 4. Enclosure 17 is made from thin non-magnetic material in order not to interfere with the inductive coupling. It is desired to make body 15 from a good magnetic material such as mild steel or silicon steel. To minimize eddy currents the cross section of body 16 in the area of coil 16 can be U-shaped or C-shaped. Plunger 18 has an internal thread 19 coupled to screw 20 connected to electric gear motor 22 and supported by thrust bearing 21. Gear motor 22 is typically a high ratio DC gear motor, preferably of the planetary type, with gear ratios in the range of 100:1 to 1000:1. Electronic module 23 connects coil 16 to gear motor 22. In the simplest form module 23 is simply a bridge rectifier. In an improved version module 23 is a bridge rectifier followed by a voltage regulator, in order to make speed of motion of actuator independent of distance to external control unit. In a different version module 23 contains a logic circuit to extract commands, such as direction reversal, from the bursts of received AC. In another version module 23 contains a battery and a program for automatic adjustment of the actuator. For example, the module can be programmed to move actuator by a fraction of a mm every day. The programming can be altered by transmitting commands from the external unit. If it is desired to send back data to external unit, module 23 can include sensors and a switching circuit to generate an AC frequency different from the actuation AC frequency and impose it on coil 17 or on a separate transmitter coil. In order to know the force and position of the actuator it is sufficient to count motor revolutions, giving position, and motor current, which is proportional to the force of plunger 18. The principles of telemetry and sensing are well known in the art of electrical engineering and need not be detailed here. An important factor in the actuator design is MRI compatibility. To achieve MRI compatibility the mass of the ferromagnetic material has to be minimized, to reduce force by the MRI static magnetic field, and any large closed loops capable of responding to high frequency should be avoided, to prevent heating by the MRI RF field. The mass of magnetic housing 15 should be minimized, as long as sufficient AC flux passes through coil 16. Since the electrical self-resonance of a typical coil needed for inductive coupling at low frequency is below 1 MHz the coil will not respond to the RF field of an MRI machine, which is typically above 60 MHz. A capacitor can be added across coil 16 or a small high frequency inductor can be added in series to further increase MRI compatibility.

The lead screw driven actuator is of the displacement type. If a constant force is desired rather than a constant displacement a spring can be added in series with actuator. The force can be controlled directly, by the motor current, or by setting the spring to the correct displacement, similar to a spring scale.

By the way of example an actuator was built and tested with the following results: A 10 mm diameter DC gear motor with a 1093:1 gear ratio (from Maxton, www.maxonmotor.com) was coupled to a 10 mm plunger via a 6 mm diameter lead screw having a 1 mm pitch. The assemble was housed in a 10 mm IDx 12 mm OD mild steel housing, slotted to reduce eddy current. Coil 16 consisted of 1000 turns of 0.25 mm diameter copper wire and hermetic housing was made of 0.2 mm thick type 316L stainless steel. Electronic module 23 was a full wave rectifier coupled to a 5VDC regulator, both surface mount type. Transmitter unit had a U-shaped laminated silicon steel core about 20 mm×20 mm×100 mm wound with 2000 turns of 0.5 mm wire and operated directly from the 120V/60 Hz mains. The actuator could generate over 250 Nt of force and could easily operate through 25 mm of tissue.

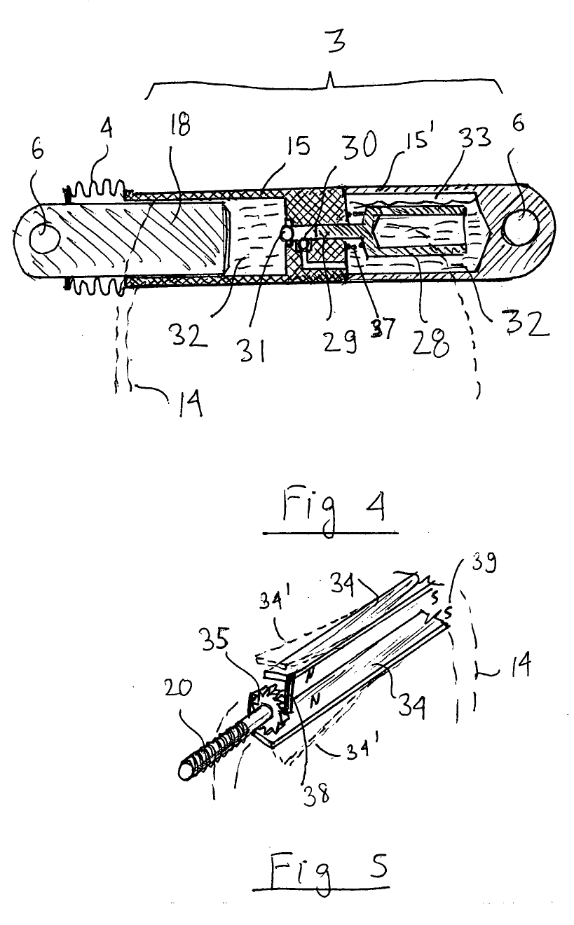

A different actuator, based on gas pressure, is shown in FIG. 2. A hermetically sealed metal enclosure 17, having a flexible metal bellows 4, is at least partially filled with a liquid 26 having a boiling point in the range of 30-50 degrees C. at atmospheric pressure. By the way of example, diethyl ether (BP=34.6 deg) and methyl n-propyl ether (BP=38.6) are suitable. The desired boiling point can be accurately adjusted by a blend of liquids. Ethers and alcohols are desirable as they are non-toxic in the case of a leak. To expand the actuator it is heated from outside the body with an induction heating coil 36. Induction heaters are commercially available and well known in the metal and wood working industries. As the actuator is heated, the liquid evaporates and expands the bellows 4 with a considerable force. As the boiling point increases with pressure, the liquid has to be heated to 30%-60% above the atmospheric pressure boiling point, or temperatures in the range of 50-75 degress C. At those temperatures a thermal insulation 27 is desired. A suitable thermal insulation is silicone rubber foam. In order to prevent the actuator from returning to original position once it has cooled down, a one-way ratcheting mechanism is used. Flexible claws 25 can move only in one direction on saw-tooth shaped rod 24. Forces of over 500 Nt can be achieved from a 3 cm diameter actuator. Still another type of actuator is shown in FIG. 4. In this actuator a pulsating DC magnetic field activates a piston pump that hydraulically moves plunger 18. When subject to magnetic field 14 ferromagnetic plunger 33, coupled to piston 29, is attracted to ferromagnetic body 15 and pressurizes fluid 32 to move plunger 18. When magnetic field 14 is removed, spring 37 retracts piston. One way valves 30 and 31 allow pumping of fluid 32 only in one direction, from reservoir 33 into part 15. Part 15′ of actuator body is made from a non-magnetic material. Fluid 32 can be a saline solution or a low boiling point liquid such as diethyl ether. Advantage of ether is that no vacuum is formed in reservoir 33, as ether will boil of to keep pressure nearly constant.

The advantage of this type of actuator is the very large forces that can be achieved, by making the ratio of plunger 18 diameter to piston 29 diameter very large, based on the well known principle of the hydraulic press. For example, by using a piston 29 having a diameter of 1 mm and a plunger 18 having a diameter of 12 mm, and using a pulsed magnetic field of 0.5 Tesla, forces over 500 Nt can be achieved. The high magnetic filed is achieved by discharging a capacitor into a coil. By the way of example, a 5000 uF capacitor charged to 200VDC and discharged into the coil of 1000 turns of 1 mm diameter wire wound on a U-shaped laminated silicon steel core will generate a sufficient field inside the tissue.

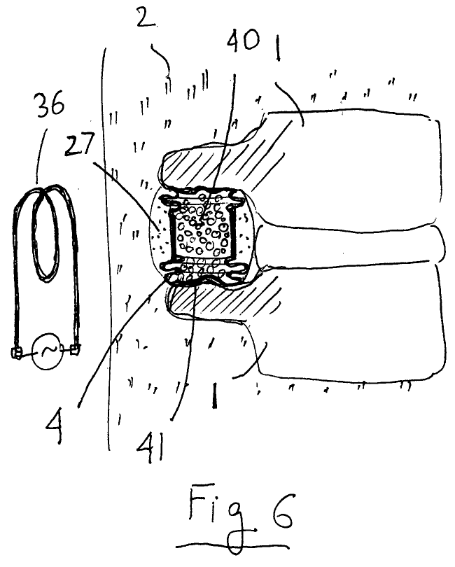

FIG. 5 shows a generic principle of an actuator based on small motions of one or more of flexible members 34. The motion can be induced magnetically, acoustically or manually, by manipulating the surrounding tissue. Flexible member 34 can turn ratchet wheel 35 by pawl. Each time member 34 deflects to position 34′ ratchet wheel 35 will advance one tooth. When members 34 are ferromagnetic and placed in a pulsed magnetic field 14 they will repel each other as they get magnetized in the same polarity 39, shown as N and S. The ratchet wheel 35 is coupled to lead screw 20 as in the motorized actuator of FIG. 2. When teeth on wheel 35 are microscopic (or a toothless one way clutch is used), members 34 can be activated by vibration from sub-sonic frequencies to ultrasonic frequencies. The operation is similar to commercial ultrasonic motors. Members 34 can also be activated by directly pressing on the skin of the patient. Typically orthopedic actuators are planted close to the surface of the body. Pressing and releasing the skin above the actuator will compress and relax member 34, rotating wheel 35. Clearly the complete actuator should be sealed in a flexible enclosure. Since this actuator contains no electrical parts or liquids, the enclosure need not be hermetically sealed and can be a saline filled polymeric bag. It can be easily made MRI compatible as it contains a minimal amount of metal parts. All other construction and sealing details are similar to other actuators in this disclosure.

In many orthopedic devices it is desired to prevent high stress point at the contact between the actuator and the bone. Since the surface of the bone can be irregular, and may change with time, it is desired for the actuator to conform to the bone surface and distribute the load evenly across the whole area of the interface. Such an actuator is shown in FIG. 6. In this example the actuator is based on heating a material 40 that can expand when heated, similar to the actuator described in FIG. 3. Instead of a ratchet mechanism a material that softens and expands when heated, but stays expanded when cooled down can be used. If a spring-like actuator is desired, any material that releases a gas when heated can be used. The material can be as simple as baking soda (NaHCO₃), which releases CO₂ gas when heated to about 60 degrees C. The pressurized gas provides a spring-like cushioning action. If a fixed size actuator is desired, the gas releasing material or the evaporating material is mixed with a rigid material that has a melting point or glass-transition point of about 42 to 100 degrees, and more desirable 45 to 55 degrees. Temperatures above 45 degrees may require a thermal insulating layer 27 to avoid tissue damage. Higher temperatures may be used as well, except heating and cooling times are longer. As the gas is released the material takes the shape of foam. When cooled down the foam becomes rigid. Clearly any combination of rigid and flexible foams can be used, depending on the desired elasticity of the actuator. Examples of low melting point rigid materials are some hard waxes and Wood's metal (an Indium alloy melting at about 70 degrees C). It is desired to add a fiber based filler such as cotton or metal fibers in order to create a more uniform structure. The filler prevents the generated gas bubbles from rising to the top. A very effective actuator can be made simply by using a stressed (compressed or extended)spring inside the sealed enclosure, with the rest of the volume filled by Wood's metal. When heated, the filler melts and the spring is free to move and take up any slack between the bones. As soon as the filler alloy solidifies, the spring is immobilized. This method allows a very strong and compact actuator. The spring may have cushioned ends to distribute the load. If some residual elasticity is desired the filler material should not fill the whole sealed volume of the actuator. The part of the spring that is not immobilized by the filler will provide elasticity.

In order to distribute the load evenly over the uneven surface of bone 2, the ends 40 of the enclosure are made of thin deformable material such as 0.2-0.5 mm thick type 316L stainless steel or thin titanium. The gas pressure inside actuator forces end plate 40 to take on the shape of the bone and distribute the load evenly. To adjust actuator from outside the body an induction heater 36 can be used. As the bone recedes over time the actuator can be expanded without surgery and it will conform to the new shape of the bone.

What is claimed is:

1. An orthopedic actuator capable of precise and continuous position control in response to a changing external magnetic field.

2. A continuously and remotely adjustable orthopedic actuator not requiring a transdermal connection.

3. An orthopedic actuator powered by a coil, said coil inductively coupled to an external coil.

4. An actuator as in claim 1 comprising a coil and an electric motor.

5. An actuator as in claim 1 comprising a hermetically sealed housing at least partially filled with a fluid.

6. An actuator as in claim 2 comprising a stressed spring immobilized by a material capable of being heated from outside the body.

7. An actuator as in claim 2 comprising a hermetically sealed housing at least partially filled with a fluid.

8. An actuator as in claim 2 capably of transmitting information from inside the body to the outside.

9. An actuator as in claim 2 capable of bi-directional operation.

10. An actuator as in claim 2 used for altering the shape of a bone.

11. An actuator as in claim 2 used for altering the gap between bones.

12. An actuator as in claim 2 capable of automatic adjustments,

13. An actuator as in claim 2 powered by ultrasonic waves.

14. An actuator as in claim 2 powered by mechanically manipulating the surrounding tissue.

15. An actuator as in claim 2 comprising a material releasing a gas when heated.

16. An actuator as in claim 1 comprising a hermetically sealed housing at least partially filled with a fluid.

17. An actuator as in claim 1 capably of transmitting information from inside the body to the outside.

18. An actuator as in claim 1 capable of bi-directional operation.

19. An actuator as in claim 2 including an element capable of adapting to the shape of the bone contacting said actuator.

20. An actuator as in claim 2, wherein said actuator comprises a sealed enclosure filled with an expandable material.
